(19) United States
(12) Patent Application Publication
(10) Pub. No.: US 2012/0165785 A1
(43) Pub. Date: Jun. 28, 2012

(54) Catheter System for a Needle Injector with an Automatic Needle/Barrier Extension

(76) Inventor: Richard A. Schatz, San Diego, CA (US)

(21) Appl. No.: 12/977,737

(22) Filed: Dec. 23, 2010

(51) Int. Cl. A61M 5/46

(52) U.S. Cl. 604/508; 604/117

(57) ABSTRACT

A catheter system includes a positioning catheter for receiving an injection needle into its lumen. The injection needle incorporates a web member mounted directly onto its shaft. As the injection needle is moved in a distal direction to exit from the lumen of the catheter, the web member is biased to transition from a folded configuration, and into a flared configuration. Specifically, this transition occurs when the injection needle is deployed more than a predetermined distance “d” beyond the distal end of the catheter. In its flared configuration, the web member is disk-shaped and is oriented perpendicular to the needle. Thus, it acts as a barrier to limit the depth of insertion of the needle into target tissue of a patient, to a depth less than “d”, and to prevent perforation of the target tissue by the catheter tip.

### FIELD OF THE INVENTION

The present invention pertains generally to injection catheters. More particularly, the present invention pertains to systems and methods for injecting fluid medicaments into myocardial tissue, and other internal tissue of a patient. The present invention is particularly, but not exclusively, useful as a system and method having a catheter-based injection needle that incorporates mechanical means to limit needle insertion into tissue to within a predetermined depth and to prevent perforation of the catheter tip through the wall of the tissue.

### BACKGROUND OF THE INVENTION

Injecting fluid medicaments into internal tissues of the body (e.g. the myocardium) can be problematic. This is particularly so due to the fact there is no way for the surgeon to have a direct visualization of the injection site. Although indirect visualization techniques, such as fluoroscopy, can significantly aid in advancing an injection needle to the intended injection site, additional control at the site may be required in order to properly insert a needle into tissue for a precise injection. For instance, control over the depth to which an injection needle is inserted into the tissue may be a crucial consideration. Further, and specific to the heart, perforation of the catheter tip through the heart wall causes blood to leak into the pericardial sack, which can be fatal.

As a practical matter, a reliance on extracorporeal control over an injection needle, for the specific purpose of precisely attaining a desired depth of needle insertion into tissue, is complicated by several factors. Not the least of these involves the proper positioning of the injection needle at the target tissue site, before needle insertion. Typically, such a pre-positioning of an injection needle can be successfully accomplished using a positioning catheter that incorporates radiopaque markers (e.g. fluoroscopy). Nevertheless, this pre-positioning relies on only indirect visual indicators that may fail to provide sufficient control for inserting the needle into target tissue.

In light of the above, it is an object of the present invention to provide a system and method for performing an injection of fluid medicament into a target tissue of a patient that provides for tactile indications of a proper needle insertion. Another object of the present invention is to ensure that such a needle insertion is performed to within a precise depth into the target tissue and to prevent perforation of the tissue by the catheter tip. Still another object of the present invention is to provide a system and method for performing an injection of fluid medicament into a target tissue that is relatively simple to manufacture, is easy to use, and is comparatively cost effective.

### SUMMARY OF THE INVENTION

In accordance with the present invention, a catheter-based injection needle is provided that controls the depth to which the needle can be inserted in an internal target tissue of a patient (e.g. the myocardium). For the purpose of controlling the insertion depth of the needle, a web member (barrier) is mounted onto the shaft of the needle at a distance “d”, proximal to the distal end of the needle. During a procedure (i.e. an injection of a fluid medicament), this web member is caused (biased) to flare outwardly from the needle. With the web member in this flared configuration, an advancement (insertion) of the needle into the target tissue is limited. Specifically, insertion of the needle is limited to the distance “d”. More specifically, this happens when the barrier makes contact with a surface of the target tissue. As envisioned for the present invention, the distance “d” can be varied according to the particular procedure being employed and the desires of the user.

Structurally, a system in accordance with the present invention includes a hollow positioning catheter having a lumen that extends between a proximal end and a distal end of the catheter. Also included in the system is the injection needle mentioned above. For purposes of the present invention, the injection needle is dimensioned to be received into the lumen of the positioning catheter for back-and-forth (proximal-and-distal) movements in the lumen. Further, the web member is dimensioned to pass through the lumen of the catheter along with the injection needle. To do this, the web member is confined by the positioning catheter to assume a folded configuration inside the lumen of the positioning catheter. While the web member is held by the positioning catheter in its folded configuration, the web member is substantially cylindrical shaped and is oriented parallel to the co-axis of the needle and the catheter. With the web member in this folded configuration, and with the positioning catheter pre-positioned in the vasculature of a patient, the injection needle can be advanced through the positioning catheter to the site of the target tissue. Alternatively, if the positioning catheter is not pre-positioned in the vasculature of that patient, the injection catheter can be advanced into the vasculature together with the positioning catheter. In either case, once the system is adjacent the target tissue site, the injection needle and web member are deployed form the distal end of the positioning catheter.

When the injection needle is deployed from the distal end of the positioning catheter, the web member is no longer constrained by the catheter, and it is biased into its flared configuration. As envisioned for the present invention, a deployment of the injection needle (web member) can be accomplished either by withdrawing the catheter in a proximal direction relative to the injection needle, or by advancing the injection needle in a distal direction relative to the catheter. Regardless how it is deployed, when it is in its flared configuration, the web member establishes a disk-shaped barrier that is oriented substantially perpendicular to the co-axis of the catheter and the needle. As indicated above, this barrier is located at the selected distance “d” from the distal end of the injection needle. As also indicated above, the purpose here is to limit the insertion depth of the injection needle to the distance “d”. Also, when deployed, the barrier acts to prevent any distal movement of the catheter beyond the barrier, to thereby prevent the catheter tip from perforating the target tissue. Once an injection has been completed, the injection needle can be withdrawn into the lumen of the catheter. Inside the lumen, the web member will again assume its folded configuration. The system can then be removed from the patient.

Several different structural arrangements for the barrier that is established by the web member in its flared configuration are envisioned for the present invention. These include an arrangement wherein the web member comprises a plurality of elongated extensions, with each extension having a first end mounted on the injection needle. For this arrangement, each extension is biased to move the opposite (second) end radially outward from the axis with a deflection of the extension. Another possible arrangement for the web member includes a plurality of interconnected straight wires. In this arrangement, a first plurality of base wires will each have an end attached to the needle. A second plurality of wires will then have each of their ends attached to a respective base wire to thereby interconnect the base wires. Also, in another arrangement, the web member may comprise a plurality of elongated wire loops. Further, for each of the web member arrangements, the barrier will have a diameter “D” in its flared configuration and, typically, “D/2” will be less than “d”. As mentioned above, however, for some procedures it may be desirable for “d” to be less than “D/2”. In other aspects of the invention, the barrier can be radiopaque and made of a material such as cobalt chromium, platinum, nitinol or stainless steel. Also, the injection needle will preferably be smaller than 18 gauge, and the variously selected distance “d” will generally be less than 10 mm.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system of the present invention shown in an intended operational environment;

FIG. 2 is a perspective view of the system with the injection needle deployed from the distal end of a positioning catheter, and with the web member biased into its flared configuration;

FIG. 3 is a perspective view of the system with the injection needle withdrawn into the lumen of the positioning catheter, and with the web member constrained by the positioning catheter into its folded configuration;

FIG. 4 is a front elevation view of an alternate embodiment of the web member; and

FIG. 5 is a front elevation view of another alternate embodiment of the web member.

### DESCRIPTION OF THE PREFERRED EMBODIMENTS

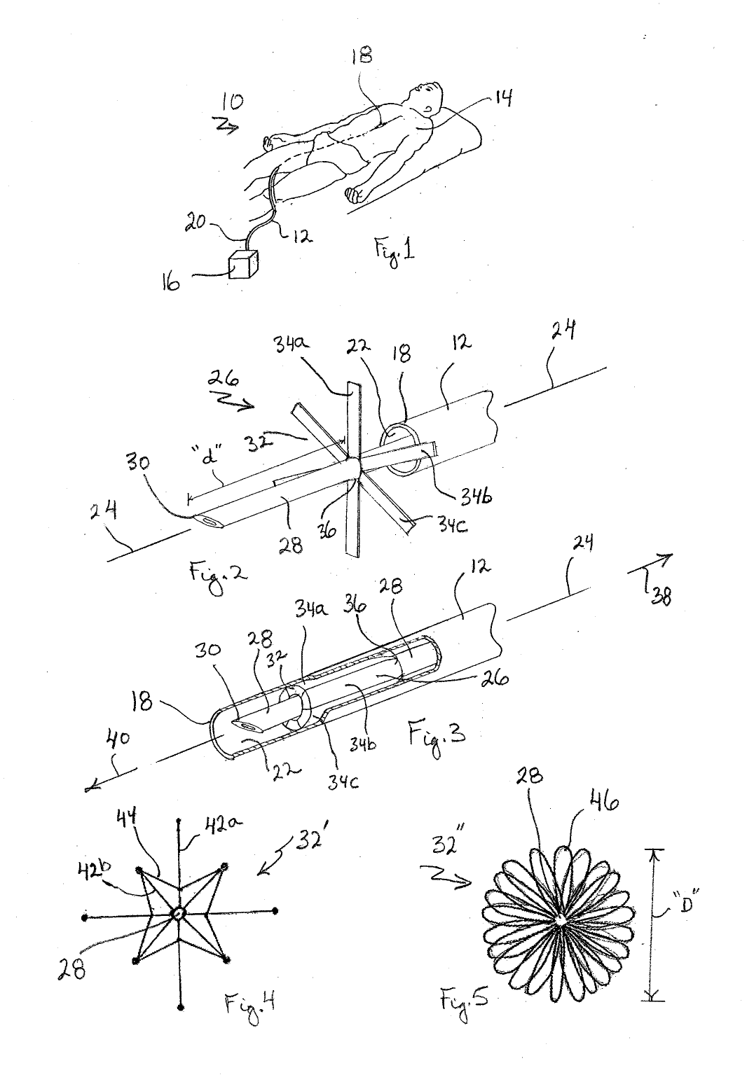

Referring initially to FIG. 1, a system in accordance with the present invention is shown in its intended operational environment and is generally designated 10. As shown, the system 10 includes a catheter 12 that can be advanced into the vasculature of a patient 14. Also, the system 10 includes a source 16 of a fluid medicament that is to be injected into an internal tissue of the patient 14 (e.g. the myocardium). For the system 10, the catheter 12 is preferably a positioning type catheter 12 having a distal end 18 and a proximal end 20, with a lumen 22 that extends along the length of the catheter 12 between the ends 18/20. As indicated in FIG. 2, the catheter 12 defines a longitudinal axis 24.

FIG. 2, shows an injection assembly for the present invention that is generally designated 26. More specifically, the injection assembly 26 includes an injection needle 28 that has a distal end 30. Preferably, the injection needle 28 is smaller than 18 gauge (e.g. 21 gauge). Additionally, the injection assembly 26 includes a web member 32 that is fixedly mounted on the injection needle 28 at a distance “d” proximal to the distal end 30 of the injection needle 28. Typically, the distance “d” will be less than ten millimeters. In other embodiments, however, the distance “d” can be adjustable. Stated differently, the exact length for distance “d” can be varied as required for the particular procedure (e.g. 3-7 mm).

For the preferred embodiment of the web member 32 shown in FIG. 2, the web member 32 includes a plurality of elongated extensions 34, of which the extension 34a, 34b and 34c are exemplary. In detail, an end of each extension 34 is affixed to the injection needle 28 at a location 36 (i.e. at the distance “d” from distal end 30). As intended for the system 10, all of the extensions 34 of web member 32 are biased to assume the position shown in FIG. 2. Specifically, each of the extensions 34 is biased to become oriented substantially perpendicular to the axis 24 when it is unrestrained. Consequently, under these unrestrained conditions, the web member 32 assumes a flared configuration that is generally disk-shaped, as shown in FIG. 2. In addition to its flared configuration, however, the web member 32 can be mechanically restrained to assume a folded configuration (see FIG. 3).

As shown in FIG. 3, when the injection assembly 26 is positioned inside the lumen 22 of catheter 12, all of the extensions 34 are forced to become individually aligned, and oriented substantially parallel to the axis 24. Under these conditions, the web member 32 becomes cylindrical shaped, to assume a folded configuration. For purposes of the present invention, it is important that the injection assembly 26 be moveable back-and-forth (i.e. proximal-and-distal) through the lumen 22 of the catheter 12, when the web member 32 is in its folded configuration.

As envisioned for the present invention, the web member 32 can be selectively transitioned between its flared configuration (FIG. 2) and its folded configuration (FIG. 3). For the system 10, this transition can be accomplished in either of two ways. For one, starting with the web member 32 in the folded configuration (FIG. 3), the catheter 12 can be withdrawn in a proximal direction (arrow 38) relative to the injection assembly 26. The consequence here is that when the distal end 30 of the injection needle 28 is more than the distance “d” from the distal end 18 of the catheter 12, the web member 32 is biased into its flared configuration (FIG. 2). For another, again starting with the web member 32 in its folded configuration, the injection assembly 26 can be advanced in a distal direction (arrow 40) relative to the catheter 12. Likewise, when the distal end 30 of the injection needle 28 is more than the distance “d’ from the distal end 18 of the catheter 12, the consequence is that the web member 32 will assume its flared configuration. To return the web member 32 from its flared configuration to its folded configuration, these operations simply need to be reversed in order to retract the injection assembly 26 into the lumen 22 of the catheter 12.

Two different alternate embodiments of the web member 32, each of which are envisioned for use with the system 10, are respectively shown in FIG. 4 and FIG. 5. In FIG. 4 an alternate embodiment of a web member 32′ is shown to include a plurality of base wires 42, and a plurality of interconnect wires 44. More specifically, for the web member 32′, each base wire 42 will have an end that is connected directly onto the injection needle 28. The interconnect wire 44, on the other hand, will have its opposite ends connected to adjacent base wires 42 (e.g. base wires 42a and 42b). In FIG. 5, the web member 32″ is shown to include a plurality of loops 46. For this embodiment, each loop 46 is connected to the injection needle 28. As shown in FIG. 4 and FIG. 5, the respective web members 32′ and 32″ are shown in their respective flared configurations. In this configuration, all embodiments (i.e. web member 32, web member 32′ and web member 32″) will establish a diameter “D” for its disk-shape. In most instances, “D/2” will be less than “d”, but it may happen that it is desirable for “d” to be less than “D/2”. Preferably, the barrier that is created by the web member 32, 32′ or 32″ is radiopaque and is made of cobalt chromium, platinum, nitinol or stainless steel.

In an operation of the system 10, the positioning catheter 12 can be pre-positioned in the vasculature of patient 14, or the injection assembly 26 can be inserted into the lumen 22 of the catheter 12 and this combination can be advanced into the vasculature. In either case, once the distal end 18 of the positioning catheter 12 is positioned at an injection site, adjacent to the target tissue (not shown), the injection assembly 26 is deployed from the catheter 12 (i.e. there is a transition of the injection assembly 26 as shown in FIG. 3 to how it is shown in FIG. 2). With this deployment, the web member 32 becomes unrestrained and is biased into its flared configuration (see FIG. 2). Thus, this flared configuration effectively establishes a barrier at the distance “d” from the distal end 30 of the injection needle 28.

With the injection assembly 26 configured as shown in FIG. 2, the injection needle 28 is inserted into the target tissue. As indicated above, the target tissue is envisioned as being either internal tissue, such as the myocardium, or external tissue like skin. Importantly, however, in each instance, the depth of this insertion into the target tissue is limited to the distance “d” by the barrier that is created when the web member 32 is biased into its flared configuration. Fluid medicament from the source 16 can then be injected into the patient 14.

Once the injection of fluid medicament has been completed, the injection needle 28 is withdrawn from the target tissue. The injection assembly 26 can then be retracted into the lumen 22 of catheter 12 until the barrier is collapsed when the web member 32 is returned to its folded configuration. The system 10 can then be removed from the patient 14.

While the particular Catheter System for a Needle Injector with an Automatic Needle/Barrier Extension as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A catheter system for performing an injection of fluid medicament into a tissue of a patient which comprises:
a catheter having a proximal end and a distal end, and defining an axis;
an injection needle positioned on the catheter, wherein the injection needle has a proximal end and a distal end; and
a means for selectively moving a web member into a flared configuration to create a disk-shaped barrier thereof, centered at a location on the injection needle, wherein the location of the barrier is at a distance “d” proximal the distal end of the injection needle and the flared configuration is established when the distal end of the needle is more than the distance “d”, in a distal direction, from the distal end of the catheter, and further wherein the web member, in its flared configuration, prevents an insertion of the needle any deeper than the distance “d” into the tissue of the patient.

2. A system as recited in claim 1 wherein the means for selectively moving the web member is the injection needle, and the flared configuration for the barrier is created when the injection needle is moved in a distal direction relative to the catheter to deploy the distal end of the injection needle beyond the distal end of the catheter.

3. A system as recited in claim 1 wherein the means for selectively moving the web member is the catheter, and the flared configuration for the barrier is created when the catheter is moved in a proximal direction relative to the injection needle to deploy the distal end of the injection needle beyond the distal end of the catheter.

4. A system as recited in claim 1 wherein the web member is biased to move from a folded configuration wherein the web member is substantially cylindrical shaped and is oriented parallel to the axis of the catheter, and into the flared configuration wherein the disk-shaped barrier is oriented perpendicular to the axis of the catheter.

5. A system as recited in claim 4 wherein the barrier has a diameter “D” in its flared configuration, and wherein “D/2” is less than “d”.

6. A system as recited in claim 1 wherein the web member comprises a plurality of elongated extensions, and wherein each extension has a first end mounted on the injection needle, and the extension is biased to move a second end radially outward from the axis with a deflection of the extension to establish the barrier.

7. A system as recited in claim 1 wherein the web member comprises a plurality of interconnected straight wires, wherein a first plurality of base wires, in the plurality of wires, each have an end attached to the needle, and a second plurality in the plurality of wires, have each end thereof attached to a respective base wire.

8. A system as recited in claim 1 wherein the web member comprises a plurality of elongated wire loops, wherein each loop is substantially oval-shaped.

9. A system as recited in claim 1 wherein the barrier is radiopaque and is made of a material selected from a group comprising cobalt chromium, platinum, nitinol and stainless steel.

10. A system as recited in claim 1 wherein the injection needle is smaller than 18 gauge, and the distance “d” is less than 10 mm.

11. A catheter system for performing an injection of fluid medicament into tissue of a patient which comprises:
a catheter having a proximal end and a distal end with a lumen extending therebetween, wherein the catheter defines an axis;
an injection needle having a proximal end and a distal end, with the injection needle received into the lumen of the catheter for alternately proximal and distal axial movements therein;
a web member mounted on the injection needle at a distance “d” from the distal end thereof, with the web member being biased to move from a first configuration wherein the web member is substantially cylindrical shaped and is oriented parallel to the axis of the catheter, and into a second configuration wherein the web member is substantially disk shaped and is oriented perpendicular to the axis of the catheter; and
a means for advancing the injection needle in the distal direction through the lumen of the catheter with the web member in its first configuration, until the distal end of the needle extends beyond the distance “d” from the distal end of the catheter to bias the web member into its second configuration for use as a barrier to prevent an insertion of the needle any deeper than the distance “d” into the tissue of the patient.

12. A system as recited in claim 11 wherein the web member has a diameter “D” when in its second configuration, and wherein “D/2” is less than “d”.

13. A system as recited in claim 11 wherein the web member comprises a plurality of elongated extensions, and wherein each extension has a first end mounted on the injection needle, and each extension is biased to move a second end thereof radially outward from the axis to deflect the extension and establish a barrier array.

14. A system as recited in claim 11 wherein the injection needle is smaller than 18 gauge.

15. A system as recited in claim 11 wherein the distance “d” is less than 10 mm.

16. A method for performing an injection of a fluid medicament into tissue of a patient, the method comprising the steps of:
positioning a catheter in the vasculature of a patient, wherein the catheter defines an axis and has a proximal end and a distal end, with a lumen extending therebetween;
advancing an injection needle through the catheter toward an injection site, wherein the injection needle is substantially coaxial with the catheter and has a proximal end and a distal end and is received in the lumen of the catheter for alternately moving in proximal and distal directions through the lumen of the catheter;
deploying the distal end of the injection needle from the distal end of the catheter at the injection site, with the distal end of the needle extending beyond the distance “d” from the distal end of the catheter;
ensuring a web member mounted on the injection needle has exited the catheter during the deploying step, wherein the web member is mounted on the injection needle at the distance “d” from the distal end of the injection needle and, during the deploying step, has moved from a first configuration wherein the web member is substantially cylindrical shaped and is oriented on the injection needle parallel to the axis of the catheter, and into a second configuration wherein the web member is substantially disk shaped and is oriented perpendicular to the axis of the catheter;
inserting the injection needle into the tissue of the patient; and
using the web member in its second configuration, during the inserting step, as a barrier to prevent an insertion of the needle any deeper than the distance “d” into the tissue of the patient.

17. A method as recited in claim 16 wherein the deploying step is accomplished by moving the injection needle in a distal direction relative to the catheter to deploy the distal end of the injection needle beyond the distal end of the catheter.

18. A method as recited in claim 16 wherein the deploying step is accomplished by moving the catheter in a proximal direction relative to the injection needle to deploy the distal end of the injection needle beyond the distal end of the catheter.

19. A method as recited in claim 16 further comprising the steps of:
withdrawing the injection needle from the tissue; and
advancing the hollow catheter in a distal direction over the injection needle to reposition the injection needle inside the lumen of the catheter with the web member returned to its folded configuration.

20. A method as recited in claim 19 further comprising the step of removing the hollow catheter and the injection needle from the patient.
